(19) United States
(12) Patent Application Publication
(10) Pub. No.: US 2020/0268426 A1
(43) Pub. Date: Aug. 27, 2020

(54) MEDICAL FIXATION DEVICE FOR POLYMER CABLES

(71) Applicant: DSM IP Assets B.V., Heerlen (NL)

(72) Inventors: William T. FISHER, Exton, PA (US); Dennis M. SAURO, Exton, PA (US); Timothy O'NEIL, Exton, PA (US)

(21) Appl. No.: 16/646,485

(22) PCT Filed: Sep. 14, 2018
(86) PCT No.: PCT/US2018/050980

§ 371 Date: Mar. 11, 2020

Related U.S. Application Data
(60) Provisional application No. 62/558,953, filed on Sep. 15, 2017.
(60) Provisional application No. 62/607,359, filed on Dec. 19, 2017.

(51) Int. Cl. A61B 17/88; A61B 17/82

(52) U.S. Cl. CPC A61B 17/8861; A61B 2017/00871; A61B 17/82

(57) ABSTRACT

Disclosed are cable fixation devices. The fixation devices are configured to keep tension in a polymer cable to secure a fracture or for other medical uses. In an embodiment, a fixation device for securing a fracture comprises a body and a piercing element, wherein the fixation device is configured to pierce a polymer cable arranged in the body by engaging the piercing element. In an embodiment, the fixation device further comprises a retensioning member, which may be operated to increase tension in the polymer cable after the polymer has been pierced by the piecing element. In an embodiment, the polymer cable comprises a braid or a yarn.

### CROSS-REFERENCE TO RELATED APPLICATIONS

This application is an international application claiming priority to U.S. Provisional Application 62/558953, filed 15 Sep. 2017, and U.S Provisional Application 62/607359, filed 19 Dec. 2017, the entire contents of each of which is hereby incorporated by reference in its entirety.

### FIELD

The present inventions relate to medical fixation devices useful with cables, kits of materials comprising fixation devices and polymer cables, and methods for securing fractures.

### BACKGROUND

Surgical cables are used in many surgical procedures to fix parts of the body, such as bones, in place. Existing cables are typically made of metal. For example, a fracture may be secured by passing a steel cable through or winding a steel cable upon a fixation device and crimping the steel cable in place to maintain tension in the cable.

Polymer cables may be preferred over steel cables because they are more pliable and generally easier to work with. A fixation device for a polymer cable is described in U.S. Pat. No. 7,207,090, assigned to Kinamed, Inc. This fixation device utilizes a body having a tapering void, and a wedge-shaped plug that is inserted into the void. When the plug is inserted in the body, a tapered channel capable of receiving a cable is defined.

### SUMMARY

It has been found that polymer cables, such as ultra high molecular weight polyethylene (UHWMPE) cables have difficulty performing in existing fixation devices designed for use with metal cables. It was found that polymer cables do not retain tension after crimping to the same degree as metal cables in existing fixation devices. Certain polymer cables are braided, which may further impact their ability to retain tension in existing devices.

Disclosed herein are fixation devices that may retain polymer cables at a higher pull-out force than prior art fixation devices, may be smaller, may be lighter, may be simpler to operate, may be cheaper, may be easier to produce, or may be otherwise more favorable than existing fixation devices.

In an embodiment, a fixation device for securing a fracture comprising a body and a piercing element, wherein the fixation device is configured to pierce a polymer cable arranged in the body by engaging the piercing element.

### BRIEF DESCRIPTION OF THE DRAWINGS

FIG. 1 is a perspective view of a first embodiment of a cable fixation device.

FIG. 2 is a perspective view of a second embodiment of a cable fixation device.

FIG. 3 is a cross-section of a third embodiment of a cable fixation device.

FIG. 4A is a side view of a fourth embodiment of a cable fixation device.

FIG. 4B is a cross-section of a fourth embodiment of a cable fixation device.

FIG. 5 is a perspective view with cutaway of a retensionable embodiment of a cable fixation device.

FIG. 6 is a perspective view of a further retensionable embodiment of a cable fixation device.

FIG. 7 is a side view of a test apparatus associated with Example 1.

### DESCRIPTION

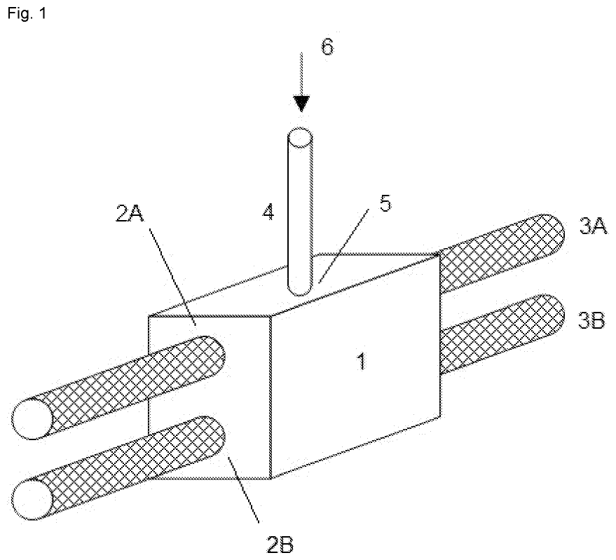

An embodiment of a fixation device is shown in FIG. 1. The device comprises a body 1. As pictured, the body is a polyhedron. The body can take any suitable shape, such as a sphere, ellipsoid, cylinder, or toroid. In an embodiment, the body is made of metal. In an embodiment, the body is made of plastic. The body has thru holes 2A and 2B though which cables 3A and 3B may be passed. In an embodiment, the body has one thru hole. In an embodiment, the body has two thru holes. In an embodiment, the body has three thru holes.

As pictured in FIG. 1, the thru holes, 2A and 2B, are straight thru holes. In an embodiment, a thru hole is straight. In an embodiment, a thru hole is curved.

The fixation device comprises piercing element 4 which is shown positioned in bore 5. The bore begins at an external surface of the body and extends through one or more thru holes. By extends through it is meant that the bore does not stop upon reaching the thru hole, but rather continues through the thru hole to the material on the other side of the thru hole. Upon passing a polymer cable through the thru hole, the body is movable along the length of the polymer cable, thereby allowing for control of its position and of the tension in the polymer cable. When the desired tension and position is reached, such that it is desired to fix the cables in position, piercing element 4 is engaged by moving the piercing element along direction 6. Upon engagement, the piercing element is configured to pierce a cable arranged in the thru hole. The piercing element and bore may be configured to pierce any number of the cables arranged in the body.

Piercing means that a portion of the polymer cable is penetrated, for instance if the piercing element possesses a smaller cross-section than the diameter of the polymer cable and penetrates through the polymer cable, or that the piercing element is able to pass through the body of the polymer cable without penetrating it, or some combination of the two. For instance, if the piercing element separates the strands of a braided cable or yarn and passes between the strands of the braided cable or yarn without penetrating or severing the strands of the polymer cable then the polymer cable is penetrated. Preferably, the piercing element does not sever any portion of the polymer cable, though the invention may still be operable if one or more strands of a braid are severed when engaging the piercing element. The piercing element may cut or sever a sheath of the cable that surrounds an interior cable, such as a braid of polymer fibers, and then pass between the strands of the braid.

The piercing element may be engaged through the cable using any suitable means, such as a crimping tool, rotational tool (such as a screwdriver), pliers, or hammer. By engaging the piercing element it is meant that the piercing element is moved sufficiently such that the piercing element would pierce a polymer cable present in the thru hole if such polymer cable was present. In an embodiment, the piercing element has a smooth surface, such as a nail or spike. In an embodiment, the piercing element is a nail. In an embodiment, the piercing element is threaded and the body comprises a complimentary threaded bore. The piercing element is engaged by turning the piercing element thereby advancing the piercing element through the bore and piercing the cable.

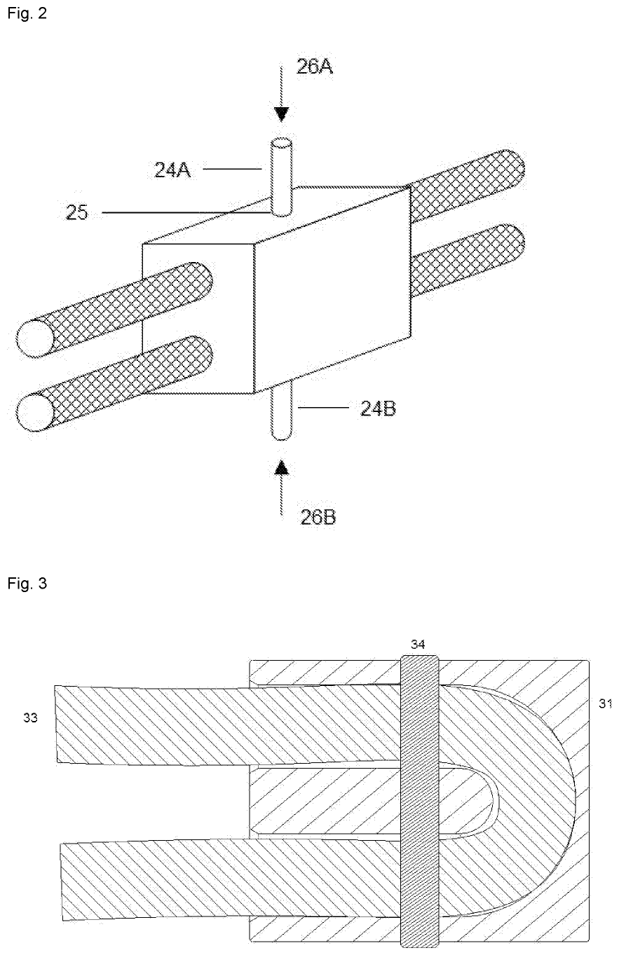

FIG. 1 pictures a single piercing element. Turning now to FIG. 2, a plurality of piercing elements 24A and 24B are shown on opposite sides of the body. The piercing elements are arranged in bore 25. Piercing element 24A may be moved in direction 26A to pierce a cable arranged in the thru hole. Piercing element 24B may be moved in direction 26B to pierce a cable arranged in the thru hole. Bore 25 extends all the way through the body such that piercing elements 24A and 24B share the same bore. In an embodiment, the piercing elements are arranged coaxially as in FIG. 2. In an embodiment, the piercing elements are not arranged coaxially, such that more than one bore is present. In an embodiment, the device comprises a plurality of bores and piercing elements arranged in the bores and the bores are not arranged coaxially.

In an embodiment, the device is configured to pierce a cable arranged in a thru hole in multiple locations. This may be done by looping a single cable through a plurality of through holes or by arranging multiple piercing elements and bores to pierce a cable in a thru hole in a plurality of locations. In an embodiment, the device comprises a plurality of bores extending through the body and piercing elements arranged in the bores, wherein the device is arranged such that when a cable is arranged in a thru hole, each piercing element pierces the cable in a different location.

FIG. 3 depicts a cross-section of an embodiment where the thru-hole is curved. The body 31 comprises a thru hole that is curved. Cable 33 may be passed along the curved thru hole such that body 31 is movable along cable 33. As pictured in FIG. 3, piercing element 34 is engaged and has pierced cable 33 in two locations. In an embodiment, the fixation device comprises a plurality of bores and associated piercing elements, allowing a cable to be pierced in at least one location per piercing element present.

In an embodiment, a fixation system comprises the fixation device and a polymer cable. In an embodiment, the polymer cable comprises polyethylene. In an embodiment, the polymer cable comprises polymer fibers. A fiber is a long continuous filament. In an embodiment, the polymer cable consists of polymer fibers. In an embodiment, the polymer cable comprises a braid of a plurality of strands of individual polymer cables or fibers. In an embodiment, the polymer cable comprises a braid that comprises a hollow tubular braid, a solid circular braid, a spiroid braid, a flat braid, a core-sheath (sometimes called kern-mantle or core-shell) braid, or a braid-on-braid. A braid-on-braid is a core-shell construction wherein a braided core is covered by another braided construction. In an embodiment, the polymer cable comprises a yarn. A yarn is a continuous strand of multiple, usually twisted, fibers. In an embodiment, the polymer cable comprises a braided, knitted, or woven cable, wherein the polymer cable comprises polymer fibers. In an embodiment, the polymer cable comprises a monofilament or a multifilament yarn. The yarn may in addition comprise other components or additives that provide some extra functional effect, such as antimicrobial or anti-inflammatory action, knotting performance, or visual contrast. In an embodiment, the polymer cable comprises a radiopaque agent.

In an embodiment, the fixation device further comprises a fastener. The fastener allows the fixation device to be fastened to a bone plate or a bone. The fastener may be complimentary to a fastener position on the bone such that the fixation device is secured to a bone when the fastener on the fixation device is secured to the complimentary fastener on the bone. In an embodiment, the fastener is a snap, a latch, a nail, or a screw.

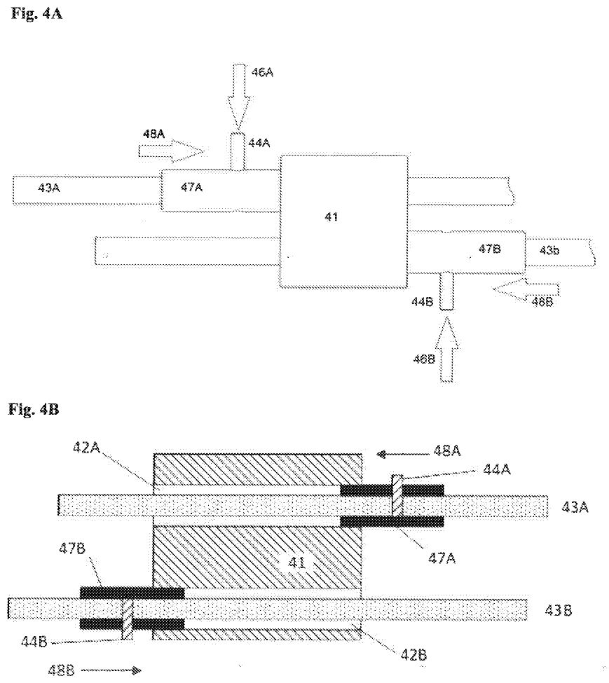

FIG. 4A depicts a side view of a further embodiment. FIG. 4B depicts a cross-section of the same embodiment. The fixation device in FIGS. 4A and 4B can receive two cables. In FIG. 4, Body 41 comprises two straight thru holes, 42A and 42B. In each thru hole, a retensioning member 47A and 47B is present arranged in the thru hole such that the retensioning member is slidable in the thru hole at least in direction 48A and 48B, respectively. In an embodiment, the retensioning member is press-fit in the thru hole. In an embodiment, the retensioning member possesses a thread that is complimentary to a thread in the thru hole. As depicted in FIGS. 4A and 4B, the retensioning members comprise the shape of hollow cylinders. The retensioning member may be in any suitable shape complimentary to the shape of the thru hole. In an embodiment, the retensioning member comprises a stopper such that the retensioning member does not accidentally fall out of the thru hole.

Although shown with two thru holes and two retensioning members, i.e. one retensioning member per thru hole, in an embodiment, a single thru hole has two retensioning members, one at each end.

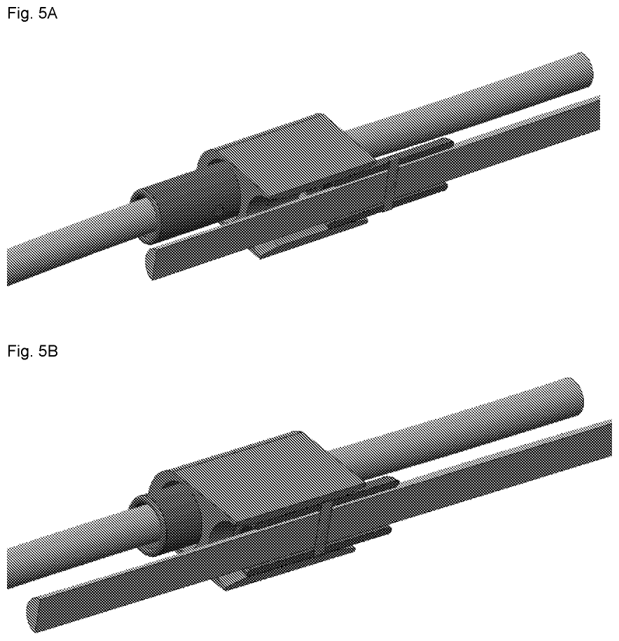

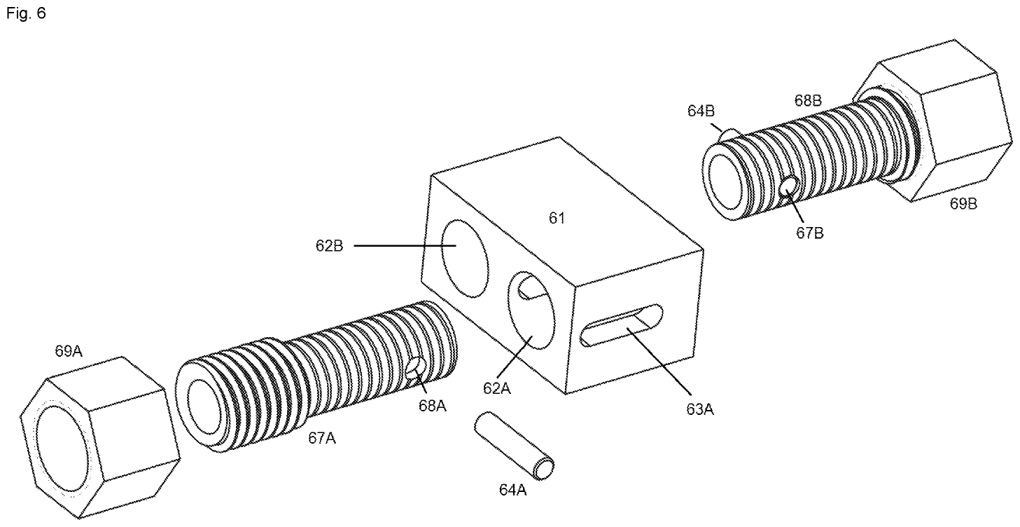

In an embodiment, the thru hole is threaded and the retensioning member possesses a complimentary thread to the thru hole, allowing retensioning to be accomplished by advancing the retensioning member along the thread. Such a device may require that less manual force is applied to retension than in a press-fit device disclosed in FIG. 4-5. Each retensioning member 47A and 47B comprises a bore and a piercing element, 44A and 44B respectively, arranged in the bore. Piercing elements 44A and 44B may be moved in direction 46A and 46B, respectively, to pierce a cable arranged in the retensioning member. The bore may pass entirely through the retensioning member or may pass only through one side. As depicted in FIGS. 4A and 4B, the bore passes through just one side of the retensioning member.

FIG. 4B depicts the fixation device of FIG. 4 in operation. Cables 43A and 43B have been passed through thru holes 42A and 42B and retensioning members 47A and 47B. The cables are tensioned and piercing elements 44A and 44B are engaged and have therefore pierced the cables 43A and 43B, respectively, holding the cables in tension. In order to retension the cables, the retensioning members 47A and 47B are slid along directions 48A and 48B, respectively, thereby increasing the tension in the polymer cable. The retension members may be slid using a suitable hand tool, such as pliers or a crimping tool.

FIGS. 5A and 5B show a further embodiment wherein the bore passes entirely through the retensioning member. The lower cable is shown cutaway. The piercing element is engaged. In the embodiment shown in FIG. 5, when the piercing element is engaged it is at least flush with the outer surface of the body. In this way, the retensioning member can be slid into the body past the location of the piercing element, as shown in FIG. 5B.

An embodiment of a further fixation device is shown in FIG. 6. In FIG. 6, a fixation device comprises a body 61. The body 61 comprises two thru holes 62A and 62B passing through the body. Thru hole 62A intersects slot 63A. The slot 63A passes from the external surface of the body 61 into the interior of the body. A slot 63B intersecting thru hole 62B and opposite from slot 63A is present in the embodiment but not visible in the view of FIG. 6. In an embodiment, the slot does not extend all the way through the body so that piercing elements on opposite sides of the body do not obstruct one another. Slots 63A and 63B are configured to receive piercing elements 64A and 64B. Piercing elements 64A and 64B may be engaged to pierce a polymer cable passing through retensioning members 67A and 67B, respectively. Retensioning members 67A and 67B include thru holes 68A and 68B, respectively, configured to receive piercing elements 64A and 64B, respectively. Retensioning members 67A and 67B are threaded complimentary to nuts 69A and 69B, respectively.

In operation, retensioning member 67A is positioned in thru hole 62A and thru hole 68A is aligned with slot 63A. Nut 68A is threaded onto retensioning member 67A such that thru hole 68A is fully visible in slot 63A, aligned with slot 63A, and at a location most distant or most proximate nut 69A, depending on the threading direction and desired direction of retensioning. A polymer cable (not pictured) may be positioned through thru hole 62A, retensioning member 67A, and nut 69A. The polymer cable may be tensioned and pierced by inserting piercing element 64A into slot 63A and thru hole 68A, thereby securing the polymer cable in place. The process may be repeated on the opposite side, involving thru hole 62B, slot 63B, piercing element 64B, retensioning member 67B, thru hole 68B, and nut 69B. Should retensioning be needed, nut 69A or 69B may be turned, thereby advancing retensioning member 67A or 67B, respectively, and increasing the tension in the polymer cable.

### Examples

A fixation device as shown in FIG. 2 (Ex. 1) and a stainless-steel crimp sleeve from Stryker Dall-Miles® 2.0 mm Beaded SS Cable Sleeve Set part # 3704-0-050 (Ex. 2) are tested with polymer cables. The fixation device in Example 1 pierces the polymer cable whereas the stainless-steel crimp sleeve in Example 2 crimps the polymer cable. The polymer cable is 2.0 mm in diameter and consists of a braid made of 176 strands of Dyneema Purity® RP 135dtex TS 100 UHWMPE fibers. For calibration, data is also included for a Dall-Miles® stainless steel crimp sleeve with the 2.0 mm stainless steel cable included in the kit (Ex. 3).

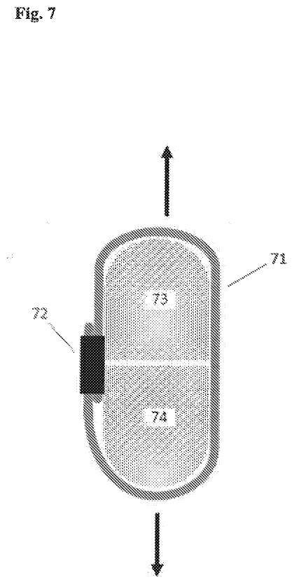

The fixation devices are tested using a Zwick Z010 tensile tester, equipped with a 10 kN load cell. Reference is made to FIG. 7. A cable 71 is looped and fixed with a fixation device 72 around two halved block grips 73 and 74 mounted on the tensile tester. A pre-tension of 2 N is applied to tension the entire set up prior to testing. A testing speed of 50 mm/min is applied in the indicated direction. This generates an increasing vertical load on the cable and fixation device. The failure mode is defined as loss of tension due to either cable slippage or breakage, which also determines the end of the test. The maximum force (Fmax) is recorded for each setup. Results are shown in Table 1.

TABLE 1
Example Results

| | Fixation | | Fmax | Failure |
|---|---|---|---|---|
| Ex. | Device | Cable | [N] | Mode |
| 1 | As shown in | Polymer | 1279 | Slippage |
| | FIG. 2 | | | |
| 2 | Dall-Miles ® SS | Polymer | 600 | Slippage |
| | Crimp Sleeve | | | |
| 3 | Dall-Miles ® SS | Stainless-Steel | 5339 | Breakage |
| | Crimp Sleeve | | | |

### Additional Description of Exemplary Embodiments

1. A fixation device for securing a fracture comprising a body and a piercing element, wherein the fixation device is configured to pierce a polymer cable arranged in the body by engaging the piercing element.
2. A fixation device for securing a fracture comprising:
   a. a body;
   b. a thru hole passing through the body;
   c. a bore passing from an external surface of the body and arranged to receive a piercing element; and
   d. a piercing element in the bore;
wherein the fixation device is configured to pierce a polymer cable arranged in the thru hole by engaging the piercing element.
3. A fixation device for securing a fracture comprising:
   a. a body;
   b. a thru hole passing through the body;
   c. a retensioning member positioned in the thru hole, arranged coaxially with the thru hole, and extending beyond the perimeter of the body;
   d. a bore passing from an external surface of the retensioning member and arranged to receive a piercing element; and
   e. a piercing element in the bore;
wherein the fixation device is configured to pierce a polymer cable arranged in the thru hole by engaging the piercing element.
4. A fixation device for securing a fracture comprising:
   a. a body;
   b. a thru hole passing through the body;
   c. a retensioning member positioned in the thru hole, the retensioning member arranged coaxially with the thru hole, and extending beyond the perimeter of the body;
   d. a bore passing from an external surface of the retensioning member and arranged to receive a piercing element;
   e. a piercing element present in the bore; and
   f. a slot passing from the exterior of the body through the thru hole and configured to constrain the piercing element in at least one direction;
wherein the fixation device is configured to pierce a polymer cable arranged in the thru hole by engaging the piercing element.
5. A fixation device for securing a fracture comprising:
   a. a body;
   b. a thru hole passing through the body;
   c. a retensioning member positioned in the thru hole and configured to be engaged by a nut, the retensioning member arranged coaxially with the thru hole, and extending beyond the perimeter of the body;
   d. a bore passing from an external surface of the retensioning member and arranged to receive a piercing element;
   e. a piercing element present in the bore; and
   f. a slot passing from the exterior of the body through the thru hole and configured to constrain the piercing element in at least one direction;
wherein the fixation device is configured to pierce a polymer cable arranged in the thru hole by engaging the piercing element, and wherein advancing the nut engaged with the retensioning member causes the piercing element to move in the slot.
6. A method of securing a fracture comprising:
   a. providing a fixation device according to any one of the previous exemplary embodiments,
   b. providing a polymer cable;
   c. stabilizing a part of a human or animal body in need of stabilization with the polymer cable;
   d. engaging the piercing element, thereby piercing the polymer cable and securing the polymer cable in place relative to the fixation device.
7. The method of securing a fracture according to any one of the previous exemplary embodiments, further comprising the step of passing the polymer cable through the body.
8. The method of securing a fracture according to any one of the previous exemplary embodiments, further comprising the steps of passing the polymer cable through the thru hole, and engaging the piercing element, thereby piercing the polymer cable and securing the polymer cable in place in the thru hole.
9. The method of securing a fracture according to any one of the previous exemplary embodiments, further comprising the steps of passing the polymer cable through the thru hole and the retensioning member, and engaging the piercing element, thereby piercing the polymer cable and securing the polymer cable in place in the thru hole.
10. The method according to any one of the previous exemplary embodiments, further comprising the step of moving the retensioning member, thereby increasing the tension in the polymer cable.
11. The method according to any one of the previous exemplary embodiments, further comprising the step of moving the retensioning member toward the body, thereby increasing the tension in the polymer cable.
12. The method according to any one of the previous exemplary embodiments, further comprising the step of moving the retensioning member away from the body, thereby increasing the tension in the polymer cable.
13. A kit for securing a fracture comprising:
   a. the fixation device according to any one of the previous exemplary embodiments; and
   b. a polymer cable capable of securing a fracture.
14. The fixation device, kit, or method of any one of the previous exemplary embodiments, wherein the thru hole is straight.
15. The fixation device, kit, or method of any one of the previous exemplary embodiments, wherein the thru hole is curved.
16. The fixation device, kit, or method of any one of the previous exemplary embodiments, wherein the piercing element, bore, and thru hole are arranged such that the piercing element pierces a cable positioned in the thru hole in one location.
17. The fixation device, kit, or method of any one of the previous exemplary embodiments, wherein the piercing element, bore, and thru hole are arranged such that the piercing element pierces a cable positioned in the thru hole in a plurality of locations.
18. The fixation device, kit, or method of any one of the previous exemplary embodiments, wherein the piercing element is a rod.
19. The fixation device, kit, or method of any one of the previous exemplary embodiments, wherein the piercing element is a spike.
20. The fixation device, kit, or method of any one of the previous exemplary embodiments, wherein the piercing element is a screw.
21. The fixation device, kit, or method of any one of the previous exemplary embodiments, further comprising a second piercing element arranged in the bore coaxially with the piercing element.
22. The fixation device, kit, or method of any one of the previous exemplary embodiments, wherein the bore extends through the thru hole.
23. The fixation device, kit, or method of any one of the previous exemplary embodiments, wherein the bore extends through the retensioning member.
24. The fixation device, kit, or method of any one of the previous exemplary embodiments, wherein the slot extends through the thru hole.
25. The fixation device, kit, or method of any one of the previous exemplary embodiments, wherein the retensioning member is slidably positioned in the thru hole.
26. The fixation device, kit, or method of any one of the previous exemplary embodiments, further comprising a second retensioning member arranged in the thru hole at the opposite end of the retensioning member.
27. The fixation device, kit, or method of any one of the previous exemplary embodiments, wherein the fixation device further comprises a second piercing element, and wherein the fixation device is configured to pierce a polymer cable arranged in the body by engaging the second piercing element.
28. The fixation device, kit, or method of any one of the previous exemplary embodiments, further comprising a second bore and a second piercing element arranged in the second bore.
29. The fixation device, kit, or method of any one of the previous exemplary embodiments, wherein the fixation device further comprises:
   e. a second thru hole passing through the body;
   f. a second bore passing from an external surface of the body and arranged to receive a second piercing element; and
   g. a second piercing element in the bore;
wherein the fixation device is configured to pierce a polymer cable arranged in the second thru hole by engaging the second piercing element.
30. The fixation device, kit, or method of any one of the previous exemplary embodiments, wherein the fixation device further comprises:
   f. a second thru hole,
   g. a second retensioning member positioned in the second thru hole, arranged coaxially with the second thru hole, and extending beyond the perimeter of the body;
   h. a second bore passing from an external surface of the second retensioning member and arranged to receive a second piercing element; and
   i. a second piercing element in the second bore;
wherein the fixation device is configured to pierce a polymer cable arranged in the second thru hole by engaging the second piercing element.
31. The fixation device, kit, or method of any one of the previous exemplary embodiments, wherein the fixation device further comprises:
   g. a second thru hole passing through the body;
   h. a second retensioning member positioned in the second thru hole, the second retensioning member arranged coaxially with the second thru hole, and extending beyond the perimeter of the body;
   i. a second bore passing from an external surface of the second retensioning member and arranged to receive a second piercing element;
   j. a second piercing element present in the second bore; and
   k. a second slot passing from the exterior of the body through the second thru hole and configured to constrain the second piercing element in at least one direction;
wherein the fixation device is configured to pierce a polymer cable arranged in the second thru hole by engaging the second piercing element.
32. The fixation device, kit, or method of any one of the previous exemplary embodiments, wherein the fixation device further comprises:
   g. a second thru hole passing through the body;
   h. a second retensioning member positioned in the second thru hole and configured to be engaged by a second nut, the second retensioning member arranged coaxially with the second thru hole, and extending beyond the perimeter of the body;
   i. a second bore passing from an external surface of the second retensioning member and arranged to receive a second piercing element;
   j. a second piercing element present in the second bore; and
   k. a second slot passing from the exterior of the body through the second thru hole and configured to constrain the second piercing element in at least one direction; and
wherein the fixation device is configured to pierce a polymer cable arranged in the second thru hole by engaging the second piercing element, and wherein advancing the second nut engaged with the second retensioning member causes the second piercing element to move in the slot.
33. The fixation device, kit, or method of any one of the previous exemplary embodiments, wherein the first piercing element and the second piercing element are arranged on opposite sides of the body.
34. The fixation device, kit, or method of any one of the previous exemplary embodiments, wherein the first retensioning member and the second retensioning member are arranged on opposite sides of the body.
35. The fixation device, kit, or method of any one of the previous exemplary embodiments, wherein the thru hole comprises a threaded surface.
36. The fixation device, kit, or method of any one of the previous exemplary embodiments, wherein the retensioning member comprises a threaded surface.
37. The fixation device, kit, or method of any one of the previous exemplary embodiments, wherein the entire outer surface of the retensioning member comprises a threaded surface.
38. The fixation device, kit, or method of any one of the previous exemplary embodiments, wherein the thru hole comprises a threaded surface and the retensioning member comprises a complimentary thread.
39. The fixation device, kit, or method of any one of the previous exemplary embodiments, wherein the fixation device further comprises a fastener on the body arranged to fasten the fixation device to a bone plate or a bone.
40. The fixation device, kit, or method of any one of the previous exemplary embodiments, wherein the piercing element is movable along a direction that is perpendicular to the direction at which the thru hole enters and/or exits the body.
41. The fixation device, kit, or method of any one of the previous exemplary embodiments, wherein the retensioning member comprises the shape of a hollow cylinder.
42. The fixation device, kit, or method of any one of the previous exemplary embodiments, wherein the retensioning member is slidable in a direction that is parallel to the direction at which the thru hole enters and/or exits the body.
43. The fixation device, kit, or method of any one of the previous exemplary embodiments, wherein the retensioning member is slidable in a direction that is perpendicular to the direction at which the thru hole enters and/or exits the body.
44. The fixation device, kit, or method of any one of the previous exemplary embodiments, wherein the retensioning members further comprise a stopper arranged to retain the retensioning member in the thru hole.
45. The fixation device, kit, or method of any one of the previous exemplary embodiments, wherein the retensioning member is press-fit in the thru hole.
46. The fixation device, kit, or method of any one of the previous exemplary embodiments, wherein slot is configured to allow the piercing element to move in the same direction as the axis of the thru hole.
47. The fixation device, kit, or method of any one of the previous exemplary embodiments, wherein the slot is configured to allow the piercing element to move in the same direction as the axis of the thru hole and substantially constrains the piercing element in other directions
48. A fixation system comprising:
   a. the fixation device of any one of the previous exemplary embodiments,
   b. a polymer cable arranged in the thru hole.
49. A fixation system comprising:
   a. the fixation device of any one of the previous exemplary embodiments,
   b. a polymer cable arranged in the body.
50. The fixation system, kit, or method of any one of the previous exemplary embodiments, wherein the polymer cable comprises polyethylene.
51. The fixation system, kit, or method of any one of the previous exemplary embodiments, wherein the polymer cable comprises polymer fibers.
52. The fixation system, kit, or method of any one of the previous exemplary embodiments, wherein the polymer cable consists of polymer fibers.
53. The fixation system, kit, or method of any one of the previous exemplary embodiments, wherein the polymer cable comprises a braid or a yarn.
54. The fixation system, kit, or method of any one of the previous exemplary embodiments, wherein the polymer cable comprises a braid of polymer fibers.
55. The fixation system, kit, or method of any one of the previous exemplary embodiments, wherein the polymer cable comprises a braid of polyethylene fibers.
56. The fixation system, kit, or method of any one of the previous exemplary embodiments, wherein the polymer cable comprises a braid of polymer cables.
57. The fixation system, kit, or method of any of the previous exemplary embodiments, wherein the polymer cable comprises a braid that comprises a hollow tubular braid, a solid circular braid, a spiroid braid, a flat braid, a core-sheath braid, or a braid-on-braid.
58. The fixation system, kit, or method of any one of the previous exemplary embodiments, wherein the polymer cable comprises a yarn.
59. The fixation system, kit, or method of any one of the previous exemplary embodiments, wherein the polymer cable comprises a braided, knitted, or woven cable, wherein the cable comprises polymer fibers.
60. The fixation system, kit, or method of any one of the previous exemplary embodiments, wherein the polymer cable comprises a monofilament or a multifilament yarn.
61. The fixation system, kit, or method of any one of the previous exemplary embodiments, wherein the polymer cable comprises a radiopaque agent.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. While certain optional features are described as embodiments of the invention, the description is meant to encompass and specifically disclose all combinations of these embodiments unless specifically indicated otherwise or physically impossible.

1-22. (canceled)

31. A fixation device for securing a fracture comprising:
a. a body;
b. a thru hole passing through the body;
c. a bore arranged to receive a piercing element, the bore beginning at an external surface of the body and extending through the thru hole, wherein the thru hole divides the bore into a proximal portion, being between the external surface of the body and the thru hole, and a distal portion, being more distal than the thru hole; and
d. a piercing element arrangeable in the bore and having a piercing element end;
wherein the fixation device is configured to pierce a polymer cable arranged in the thru hole by advancing the piercing element end in the bore from the proximal portion of the bore into the distal portion of the bore.

32. The fixation device according to claim 31, wherein the fixation device further comprises:
e. a retensioning member positioned in the thru hole, arranged coaxially with the thru hole, and extending beyond the perimeter of the body; and
wherein the bore extends through the retensioning member, and
wherein the retensioning member is configured to increase tension in a polymer cable after initially tensioning and piercing the polymer cable.

33. The fixation device according to claim 32, wherein the bore has a first shape in the body, and a second shape in the retensioning member, and wherein the first shape is different than the second shape.

34. A fixation device for securing a fracture comprising:
a. a body;
b. a thru hole passing through the body;
c. a retensioning member positioned in the thru hole, the retensioning member arranged coaxially with the thru hole, and extending beyond the perimeter of the body;
d. a bore passing through at least a portion of the retensioning member and arranged to receive a piercing element;
e. a piercing element arrangeable in the bore and comprising a piercing element end; and
f. a slot beginning at an external surface of the body and extending to at least the thru hole, the slot configured to constrain the piercing element in at least one direction;
wherein the fixation device is configured to pierce a polymer cable arranged in the thru hole by moving the piercing element through the slot and the bore, and
wherein the retensioning member is configured to increase tension in a polymer cable after the polymer cable has been initially tensioned and pierced.

35. The fixation device according to claim 34, wherein the slot extends through the thru hole, and wherein the thru hole divides the slot into a proximal portion, being between the external surface of the body and the thru hole, and a distal portion, being more distal than the thru hole.

36. The fixation device according to claim 35, wherein the fixation device is configured to pierce a polymer cable arranged in the thru hole by advancing the end of the piercing element in the slot through the bore, past the thru hole, and into the distal portion of the slot.

37. The fixation device according to claim 34, wherein the retensioning member is configured to be engaged by a nut, and wherein advancing the nut engaged with the retensioning member causes the piercing element to move in the slot.

38. The fixation device according to claim 34, wherein the retenioning member is configured to increase tension by translating the retensioning member in the direction of the axis of the thru hole.

39. The fixation device of claim 34, wherein the slot extends through the thru hole and the bore extends through the retensioning member.

40. The fixation device according to claim 34, wherein the fixation device further comprises:
g. a second thru hole passing through the body;
h. a second retensioning member positioned in the second thru hole, the second retensioning member arranged coaxially with the second dim hole, and extending beyond the perimeter of the body;
i. a second bore passing through at least a portion of the retensioning member and arranged to receive a second piercing element;
j. a second piercing element arrangeable in the second bore and comprising a second piercing element end; and
k. a second slot beginning at an external surface of the body and extending to at least the second thru hole, the second slot configured to constrain the second piercing element in at least one direction;
wherein the fixation device is configured to pierce a polymer cable arranged in the second thru hole by moving the second piercing element arranged in the second slot through the second bore, and
wherein the second retensioning member is configured to increase tension in a polymer cable after the polymer cable has been initially tensioned and pierced.

41. The fixation device of claim 40, wherein the first retensioning member and the second retensioning member are arranged on opposite sides of the body.

42. The fixation device of claim 34, wherein the fixation device further comprises a fastener on the body arranged to fasten the fixation device to a bone plate or a bone.

43. The fixation device of claim 34, wherein the retensioning member comprises the shape of a hollow cylinder.

44. The fixation device of claim 34, wherein the thru hole extends straight through the body and the retensioning member is slidable in a direction that is parallel to the direction at which the thru hole enters the body.

45. The fixation device of claim 34, wherein the thru hole extends straight through the body, and the slot is configured to allow the piercing element to move in the same direction as the axis of the thru hole and substantially constrains the piercing element in other directions.

46. A method of securing a fracture comprising:
a. providing a fixation device according to claim 34,
b. providing a polymer cable;
c. stabilizing a part of a human or animal body in need of stabilization with the polymer cable;
d. passing the polymer cable through the body via the thru hole; and
e. engaging the piercing element, thereby piercing the polymer cable and securing the polymer cable in place.

47. The method according to claim 46, further comprising the step of moving the retensioning member, thereby increasing the tension in the polymer cable.

48. The method according to claim 46, wherein the polymer cable comprises a braid or a yarn of polymer fibers and wherein the piercing of the polymer cable is performed by passing the piercing element through the braid or yarn of polymer fibers.

49. A kit for securing a fracture comprising:
a. the fixation device according to claim 34; and
b. a polymer cable capable of securing a fracture.

50. The kit according to claim 49, wherein the polymercable comprises a braid of polymer fibers or a yarn of polymer fibers.
